(19) United States
(12) Patent Application Publication
(10) Pub. No.: US 2007/0246505 A1
(43) Pub. Date: Oct. 25, 2007

(54) SURGICAL BUTTRESS ASSEMBLIES AND METHODS OF USES THEREOF

(75) Inventors: Mark Pace-Floridia, Delta (CA); Paul Geyer, Burnaby (CA); Carlos Vonderwalde, Richmond (CA); Kevin McKim, Richmond (CA)

Correspondence Address: FASKEN MARTINEAU DUMOULIN, LLP, 2100 - 1075 WEST GEORGIA STREET, VANCOUVER, BC, V6E-3G2, US

(73) Assignee: Medical Ventures Inc., Richmond (CA)

(21) Appl. No.: 11/738,916

(22) Filed: Apr. 23, 2007

Related U.S. Application Data
(60) Provisional application No. 60/794,130, filed on Apr. 24, 2006.
(60) Provisional application No. 60/815,848, filed on Jun. 23, 2006.
(60) Provisional application No. 60/879,366, filed on Jan. 9, 2007.

(51) Int. Cl. A61B 17/10

(52) U.S. Cl. 227/175.1

(57) ABSTRACT

A surgical buttress-dispensing assembly configured for releasably engaging a surgical stapling device. The surgical buttress-dispensing assembly comprises an one-piece cartridge for receiving and engaging therein an elongate buttress strip, and a carrier configured to slidingly receive, engage, and discharge the cartridge. The cartridge comprises a cylindrical end portion from which extends a pair of opposed elongate semi-circular sleeves. The cylindrical end portion is configured to slidingly received therethrough the closed closed jaws of a surgical stapler. The semi-circular sleeves are configured such that each sleeve slidingly communicates and cooperates with one of the stapler's jaws. The elongate edges of the sleeves are configured to engage and retain therewith the elongate edges of a surgical buttress strip. The carrier is engaged with a cartridge having a buttress strip installed onto the opposed sleeves, and is manipulated to slide and mount the cartridge onto the jaws of a surgical stapler.

### CROSS-REFERENCE TO RELATED APPLICATIONS

This application claims priority from our prior provisional applications Ser. Nos. 60/794,130 filed Apr. 24, 2006; 60/815,848 filed Jun. 23, 2006; and 60/879,366 filed Jan. 9, 2007.

### FIELD OF THE INVENTION

This invention relates to surgical buttress assemblies. More particularly, this invention relates to surgical buttress assemblies configured for cooperating with surgical stapler devices.

### BACKGROUND OF THE INVENTION

Surgical stapler devices and systems are now commonly employed in most surgical procedures due to their ease-of-use, rapid action, and provision of uniformly spaced-apart sutures. There are three main types of surgical stapler devices currently in common use: (1) endoscopic stapler devices which are sized and configured for cooperation with trocar devices during laparoscopic surgical procedures, (2) linear stapler devices that are sized and configured for opened-body procedures, and (3) circular stapler devices configured for conjoining surgically-separated cylindrical organs and tissues such as small intestines and colons. Such surgical stapler devices usually have an ergonomically shaped, ambidextrous handle containing a plurality of levers and typically comprise a long shaft at the end of which is provided a fixed jaw onto which is placed a cartridge containing at least two rows of a plurality of opposed surgical staples. The fixed jaw is interconnected at a fulcrum point with a moveable jaw fitted with an anvil component. A knife component for surgically separating tissues is usually provided interposed the rows of surgical staples. Manipulation of a first actuating lever attached to the handle of the stapler, compresses the tissue to be surgically separated, after which manipulation of a second lever: (a) fires the staples contained in the cartridge against the anvil thereby surgically engaging a separate tissue section with each row of staples, then (b) actuates a knife to surgically separate the tissue sandwiched between the two rows of engaged staples. Although such surgical stapler devices and stapling systems have significantly simplified surgical procedures, a significant problem is the imposition of stretching tension and stress on a tissue section that has been engaged by a row of surgical staples. Such stretching tensions and stresses quite often result in the tissue tearing about the staple engagement points and in severe instances, may result in the conjoined tissue separating causing post-operative bleeding and other complications requiring an other episode of surgical intervention. Consequently, there have been numerous devices and methods developed for reinforcing and/or buttressing surgical stapled sutures, as exemplified by:

U.S. Pat. No. 6,939,358 which discloses a self-adherent synthetic biocompatible material which is attached to an operational surface of a surgical stapler by an application card provided with pre-cut tear lines that allow the material to be applied held in place on the stapler while the surgical procedure is carried out, and then to buttress the surgical suture lines;

U.S. Pat. No. 5,810,855 which discloses a synthetic sleeve known as “SEAMGUARD®” (registered trade mark of W.L. Gore & Associates Inc., Newark, Del., USA) which surrounds the stapler jaws provided at the end of the operational arm. After the staples have been fired and the conjoined tissue sections severed and reinforced by the SEAMGUARD® material, the excess material must be trimmed from the tissue sections and removed from the trocar before completing the surgical procedure;

U.S. Pat. No. 5,810,855 which discloses a manipulated bovine pericardium strip known PERI-STRIPS DRY® (registered trade mark of Bio-Vascular Inc., St. Paul, Minn., USA) for securing to the jaws of a surgical stapler with a biocompatible glue;

U.S. Pat. No. 6,656,193 which discloses several buttress devices configured to engage surgical stapler jaw ends. These devices are configured for mechanical retention to the jaws until the stapling procedure has been completed;

U.S. Pat. No. 6,656,193 which discloses a pericardial buttress strip provided with at least one end having an aperture for engaging at least on jaw end of the stapler; and

U.S. Pat. No. 6,704,210 which discloses a sealing film strip attached to a surgical stapler by passing a jaw of the stapler though openings formed in the ends of the strip.

Current state-of-the art surgical stapler devices typically require complex loading and securing processes to fix and maintain the buttress materials in place until the staples are fired. Consequently, there are numerous problems associated with the prior art buttress strips with such stapler devices. For example, buttress strips concurrently secured with adhesive materials to the cartridge on the fixed jaw and the anvil on the articulating jaw the stapler, require both surfaces to be completely dry in order to provide the desired orientation during staple firing. If those surfaces are not dry during the staple firing process, the buttress material will slide around on the cartridge and anvil while the user approximates the device around the tissue to be conjoined and severed. The jaws of multiple-fire staplers, i.e., stapler devices that are configured to conjoin and staple together several tissue sections in sequence typically become wet as a result of contact with tissues during the first staple firing process, thereby causing loosening the remaining buttress strips on the jaw surfaces resulting in misalignment during the second and third staple firings. Quite often, sutured tissue sections resulting from second and third staple firings are provided with partial buttressing or may not have any buttressing materials reinforcing the suture line. Another problem with the current prior art buttress materials is that the adhesive strength of the biocompatible glues used to fix the strips to the jaws is insufficient to maintain the strips in place on the jaws as the stapler is manipulated through the trocar and about the tissues to be surgically conjoined and severed. This results in considerable inconvenience to the operator since the dislodged strips need to be retrieved and re-affixed to the stapler prior to firing the staples.

Another method of attaching buttress materials to surgical stapler jaws is the use of suture threads to tie the strips to the stapler jaws; however this process is time consuming and the requisite delicate handling and precise manipulation of the stapler and buttress strips is a distraction during a surgical procedure. Another prior art method for securing buttress materials onto surgical staplers involves the use of retainer appliances configured for cooperating with the stapler jaws. However, such appliances typically add significant girth to the overall profile of the jaw portion of the stapler shaft which requires the use of larger diameter trocars to facilitate entry of the stapler device into a body cavity wherein the endoscopic surgical procedure is to be performed.

In order to minimize the size of the hole in the abdomen wall required to insert the trocar perform the endoscopic surgery and to minimize the leakage of gas and/or fluid through the trocar, the inner and outer diameters of the trocar must be configured as small as possible, while providing sufficient room for passage of the stapler device through the passageway into the body cavity. Only nominal clearance is provided for passage of the stapler device through the trocar. Therefore, the addition of buttress materials in the form of sleeves surrounding the jaws of the stapler increases the overall diameter of the stapler such that it can no longer pass through a preferred small-diameter trocar. Physicians are reluctant to use larger trocars, which provide more clearance for the above stated reasons. Ideally the buttressing material can be added to the stapler with little or no increase in diameter of the stapler such that the stapler can pass easily through a standard trocar intended for use with that stapler.

Surgical circular staplers have been developed and are commonly used for surgical joining of separate hollow organ sections into one elongate member, e.g., intestinal tissues. A buttress is commonly placed between the stapling surface and the native hollow sections that are to be joined after which one or more rows of staples are fired to sealingly connect the organ sections while simulataneously cutting out the centre of the buttress. A serious problem often encountered with these types of procedures is leakage from anastomoses resulting in loss of luminal contents from the organ tissues into the surrounding body cavities resulting in life-threatening infections. U.S. Pat. No. 6,656,193 discloses a hot melt adhesive system configured to releasably engage pericardium strips mounted onto removable perforated buttress rings which cooperate with the anvil head sections of surgical circular staplers. However, the process of fixing the buttress materials onto the buttress rings, and then engaging the loaded buttress rings with the anvil components is complicated and requires precisely controlled dexterity to ensure proper alignment of the buttress materials in the circular staplers. It is essential that the surfaces of the buttress materials and buttress rings are completely dry in order to ensure good adhesion during loading of the anvil section, and then during insertion and maneuvering of the surgical stapler into and within the body cavity. The buttress materials are often dislodged from the buttress rings during the insertion and manipulation of the stapler with the consequence that the stapler must be removed from the body cavity, the anvil section disassembled to reposition and re-adhere the buttress material before proceeding. Furthermore, it is difficult to center and maintain the buttress material in a concentric orientation with the stapler with the result that the buttress materials do not completely cover the staple line sections resulting in stapled tissue sections that are not buttressed—these are the sections predisposed to leakage of luminal materials passing therethrough.

Therefore, a need still exists for a method of easily attaching a buttress material to the stapler jaws that does not increase the outer diameter of the stapler so that it will not pass through the trocar, and finally that will secure the material to the stapler jaws even when the user approximates around the tissue.

### SUMMARY OF THE INVENTION

The exemplary embodiments of the present invention, at least in preferred forms, are directed to surgical buttress assemblies for releasably engaging surgical stapling devices configured to slidingly communicate and cooperate with trocars.

According to one embodiment of the present invention, there are provided surgical buttress assemblies comprising a cartridge configured for receiving and engaging therein an elongate buttress strip, and a carrier configured for demountably engaging said cartridge for delivery and installation onto the jaws of a surgical stapler.

According to one aspect, the cartridge comprises a cylindrical base portion from which extend two elongate opposing semi-circular sleeves. The sleeves are configured to slidingly communicate and cooperate with the outer surfaces of the jaws of a surgical stapler.

According to another aspect, the elongate side edges of each semi-circular sleeve are provided with gripping means for engaging the elongate side edges of buttress strips. Suitable gripping means are exemplified by serrated edges, resilient fingers, Velcro® strips (Velcro is a registered trademark of Velcro Industries B.V.), and forward-extending tabs. Forward-facing tabs comprising the gripping means, may be optionally provided with pointed and/or sharpened leading edges to facilitate piercing and penetration of the tabs into the edges of the buttress strips.

According to a yet another aspect, the cartridge comprises a cylindrical base portion from which extend two opposing fingers. Each finger comprises an elongate arched member from which opposing channels extend upward and outward along the longitudinal axis. Each channel is configured for receiving and engaging therein a portion of an elongate buttress strip. It is optional to provide each channel with a gripping means for engaging and retaining the elongate edges of a buttress strip. The jaws of a surgical stapler are inserted through the cylindrical base portion after which one of the elongate arched fingers slidingly communicates with the outer surface of one jaw, while the other elongate arched finger slidlingly communicates with the outer surface of the other jaw. A buttress guide is optionally provided for engaging a portion of the buttress strip interposed the two fingers during installation of the cartridge onto the surgical stapler.

According to another aspect, the cartridge comprises an elongate arched finger from which opposing channels extend upward and outward along the longitudinal axis. Each channel is configured for receiving and engaging therein a portion of an elongate buttress strip. It is optional to provide each channel with a gripping means for engaging and retaining the elongate edges of a buttress strip. A first such cartridge is engaged with one end of an elongate buttress strip, and a second such cartridge is engaged with the other end of the buttress strip. The first cartridge is then slid over one jaw of a surgical stapler with the jaw interposed the cartridge and the buttress strip. The second cartridge is slid over the other jaw of the surgical stapler with the jaw interposed the cartridge and the buttress strip. A buttress guide is optionally provided for engaging a portion of the buttress strip interposed the two cartridges during installation onto the surgical stapler.

According to yet a further preferred embodiment of the present invention, there is provided a surgical buttress apparatus for cooperating with a surgical circular stapler. The apparatus comprises a biocompatible buttress material, a buttress support disc configured for slidingly cooperating with the anvil head assembly of a circular stapler, and a device for attaching the buttress material to the buttress support disc. It is preferred that the buttress support disc is circular and is provided with a concentric aperture about the middle of the disc. It is further preferred that the aperture of the support disc is configured to sliding communicate with the shaft of the anvil head assembly of a circular stapler. The apparatus is assembled by threadably attaching the buttress material to the buttress support disc. It is preferable that that the middle portion of the buttress material is provided with an aperture that approximates the aperture of the support disc. If so desired, the buttress material may be provided with at least one slit extending inward from the aperture to facilitate use of the buttress material with circular staplers provided with anvil shafts having different diameters.

According to yet another further embodiment of the invention, there is provided a surgical buttress assembly configured for cooperating with a surgical circular stapler. The assembly comprises a biocompatible buttress material threadably attached to a circular buttress support disc configured to slidingly communicate with the shaft of the anvil head assembly of a surgical circular stapler.

### BRIEF DESCRIPTION OF THE DRAWINGS

The present invention will be described in conjunction with reference to the following drawings, in which:

FIG. 1 is a perspective view of an exemplary embodiment of the surgical buttress assembly present invention showing the cartridge and carrier components disengaged;

FIG. 2a is another perspective view of the embodiment shown in FIG. 1 showing a buttress strip interposed the cartridge and carrier components;

FIG. 2b is a perspective view showing the buttress strip installed on the cartridge, and the loaded cartridge engaged with the carrier, and ready for loading onto the jaws of a surgical stapler;

FIG. 3 is a perspective view showing a preferred orientation for installing a loaded cartridge onto the jaws of a surgical stapler;

FIG. 4a is a perspective view of the loaded cartridge from FIG. 3 installed onto the jaws of a surgical stapler'

FIG. 4b is a side view of the installed loaded cartridge from FIG. 4a;

FIG. 5 is a cross-sectional end view of another exemplary embodiment of the cartridge of the present invention shown engaged with a buttress strip, and ready for loading onto a surgical stapler;

FIGS. 6(a)-6(e) are close-up cross-sectional partial end views of exemplary gripping edges and fingers of the cartridge from FIG. 5;

FIG. 7a is a side view of a prior art surgical circular stapler provided with a detachable anvil head assembly;

FIG. 7b is a close-up view of the anvil head assembly of the surgical circular stapler shown in FIG. 4;

FIG. 8 is a perspective view of another exemplary embodiment of the present invention configured to cooperate with a surgical circular stapler; and

FIG. 9 is a close-up exploded perspective view of a prior art anvil head assembly cooperating with the embodiment shown in FIG. 5.

### DETAILED DESCRIPTION OF THE INVENTION

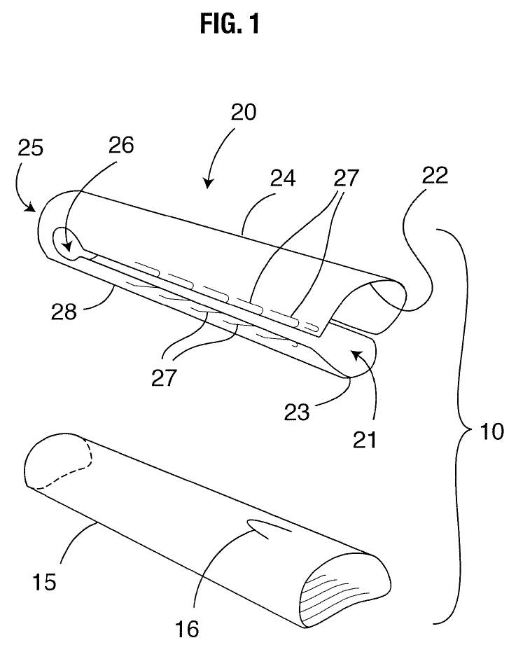

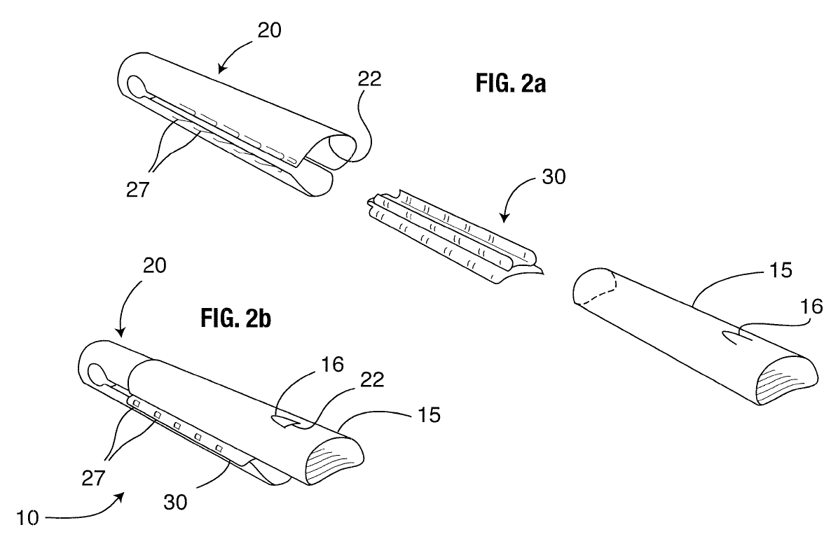

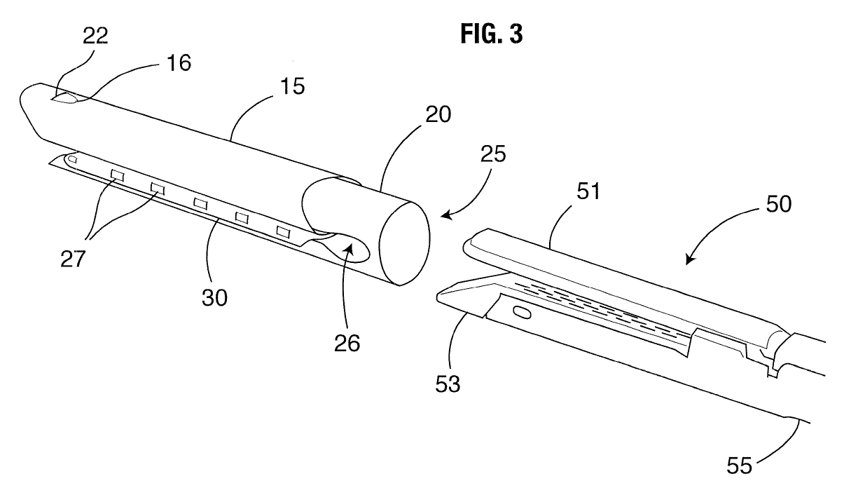

An exemplary preferred embodiment of a surgical buttress-dispensing assembly of the present invention is shown in accompanying FIGS. 1-4 and is generally referred to by the numeral 10. The surgical buttress-dispensing assembly 10 comprises a cartridge 20 and a carrier 15. The cartridge 20 is generally elongate and cylindrically shaped with an anterior end 21 and a posterior end 25. An aperture 26 is provided approximate the posterior end 25 of the cartridge 20. Two opposing channels bisecting the cartridge 20, extend forward from the aperture 26 through to the anterior end 25 of the cartridge 20 thereby providing an upper half 24 separated from the lower half 28 of the cartridge 20 anterior of the aperture 26. The upper half 24 terminates at leading edge 22 while the lower half 28 terminates at leading edge 23. The opposing side edges of the upper and lower halves 24, 28 are preferably integrally provided with forward-facing tabs 27 configured to communicate with and engage the side edges of a suitable buttress strip 30. The carrier 15 generally comprises an elongate semi-circular hollow tube provided with a backward-facing tang 16 approximate one end of the carrier 15. As best seen in FIGS. 2b and 3, the tang 16 is configured to receive and releasingly engage therein the leading edge 22 of the upper half 24 of cartridge 20, or alternatively, the leading edge 23 of lower half 28 of cartridge 20. It is preferred that the cartridge 20 and carrier 15 are made of suitably thin and flexible but durable and resilient materials as exemplified by biocompatible plastics materials known to those skilled in these arts.

As shown in FIGS. 2a and 2b, the carrier 15 may be used to install and engage a buttress strip 30 onto the cartridge by folding the buttress strip 30 approximately in half and then sliding the carrier 15 into the folded buttress strip 30 so that one end of the strip 30 is within the carrier 15 while the other end of the strip 30 overlies the outer surface of the carrier 15. The carrier 15 is then slid and manipulated over one of the halves (e.g., in FIG. 2a, the upper half 24) of the cartridge 20 thereby engaging the side edges of the buttress strip 30 with the tabs 27 on the upper and lower halves 24, 28 of the cartridge 20 until the leading edge (e.g. leading edge 22 on upper half 24 per FIG. 2a) engages the tang 16 of the carrier 16. The loaded surgical buttress assembly is now ready for installation onto the articulable jaws 51, 53 of a surgical stapler 50.

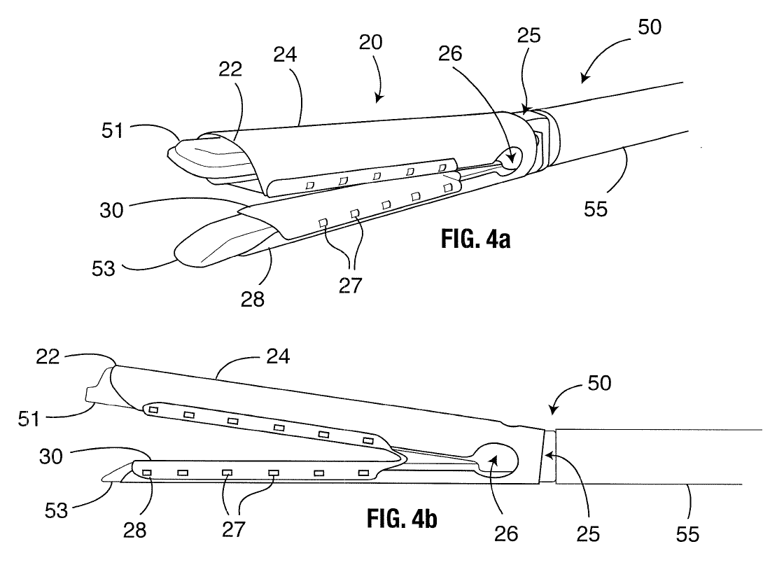

As shown in FIGS. 3, 4a and 4b, the posterior end 25 of the cartridge 20 is configured so that it may slidingly receive therein and therethrough the closed jaws 51, 53 of a surgical stapler 50. By grasping and manipulating the carrier 15, the loaded surgical buttress assembly 10 is slid onto the closed jaws 51, 53 to about the proximal end of the jaws 51, 53 after which the carrier 15 can be retracted and removed from the stapler 50. After installation of the loaded cartridge 20, the upper jaw 51 of the stapler 50 is interposed the upper half 24 of the cartridge 20 and the buttress strip 30, while the lower jaw 53 is interposed the lower half 28 of the cartridge 20 and the buttress strip 30. It is preferable to install the loaded cartridge 20 onto the stapler 50 so that the aperture 26 is approximate the proximal end of the jaws 51, 53. After such installation, the upper half 24 of the cartridge communicates and cooperates with the upper jaw 51 while the lower half 28 of the cartridge 20 communicates and cooperates with the lower jaw 53, to securely retain the buttress strip 30 in a preferred installed position during insertion of the surgical stapler 50 through a trocar (not shown) into a patient's abdominal cavity, after the jaws 51,53 have been opened and are manipulated within and about the abdominal cavity to a desired position, and during the staple-firing process. After staples have been fired by the surgical stapler 50, the buttress strip 30 is separated from the cartridge 20 by severing adjacent to the inner walls of the upper and lower halves 24, 28 of the cartridge 20, with blades that are integral components of the surgical stapler 50. The cartridge 20 is retained by the stapler jaws 51, 53 and is removed from the patient's abdomen through the trocar with the stapler 50.

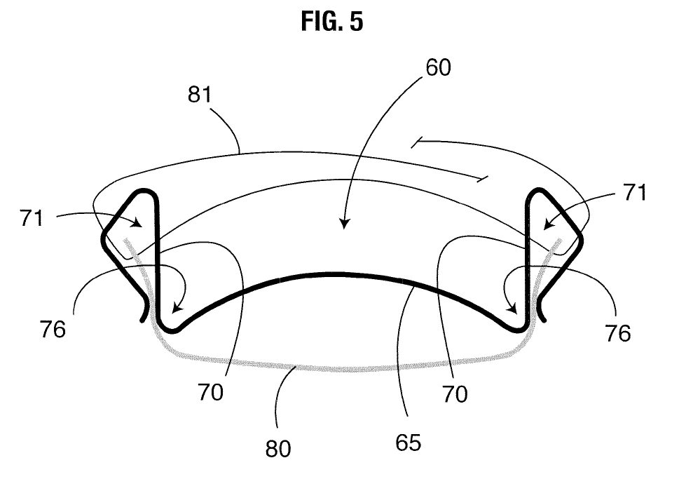

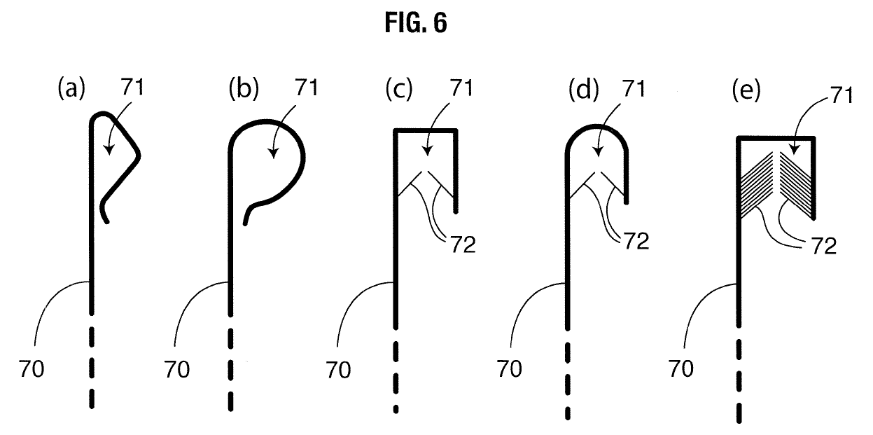

FIGS. 5 and 6 exemplify another suitable elongate cylindrical cartridge having an anterior end and a posterior end, and generally configured with an aperture approximate the posterior end from which extends forward an upper half separated from the lower half wherein the upper and lower halves are separated by a pair of opposing channels bisecting the cartridge. As exemplified by an upper half portion 60 shown in FIG. 5, the opposing elongate side edges 70 of the upper elongate cartridge partial wall portion 65 are formed to provide clamping means whereby a first pair of upward-facing channels 76 is formed by folding side edges 70 to extend upwards from the cartridge partial wall portion 65 and then a second pair of downward-facing channels 71 is formed by folding the upward extending side edges 70 outward and downward. As shown in FIGS. 6(a) and 6(b) the downward portions 71 of the opposing side edges 70 can be made to bias toward the upward extending portions of the side edges 70 thereby providing clamping means between the upward and downward portions of side edges 70. The downward-facing channels 71 may be optionally provided with at least one pair of opposed inwardly-orientated gripping edges or fingers 72 as shown in FIGS. 6(c)-6(e). The lower half of the cartridge partial wall (not shown) is configured into a mirror-image of the upper half 60 shown in FIG. 5. As shown in FIG. 5, about one half of the length of a suitable buttress strip 80 is installed into the upper half of the cartridge partial wall 60 by inserting its opposing elongate side edges into the opposing downward-facing chambers 71 in between the upward-extending and downward-extending elongate side edges 70. The other half of the buttress strip is inserted in between the corresponding downward-extending and upward-extending elongate side edges of the lower half of the cartridge partial wall. If so desired, the buttress strip 80 may be additionally secured to the cartridge after installation, with a lacing suture 81 as shown in FIG. 5. However, the biasing channels 71 will be sufficient to retain in position an installed buttress 80 on this exemplary cartridge during its delivery to the jaws of a surgical stapler and subsequent use.

Examples of suitable buttress strips comprise biocompatible materials exemplified by synthetic base materials such as expanded polytetrafluoroethylene (ePTFE), VICRYL® (registered trade mark of Johnson and Johnson Corp., New Brunswick, N.J., USA) which is a periodontal mesh prepared from bioabsorbable copolymers derived from glycolic acid and lactic acid, DEXON® (registered trade mark of Sherwood Services AG Corp, Schaffhausen, Swizterland) which is a polyglycolic acid, and TEFLON® (registered trade mark of E.I. DuPont de Nemours and Co., Wilmington, Del., USA) which is a polytetrafluoroethylene, and collagen-absorbable hemostat, and stabilized naturally occurring materials such as a pericardium material, and other such materials.

The exemplary embodiments of the surgical buttress assemblies of the present invention disclosed herein are designed to provide secure sealing of surgical staple lines produced by linear cutting types of staplers, by the precise manipulation and application of biocompatible materials within visceral host tissue for use as buttressing materials for suture lines. The system provides a “sandwich” effect by placing a reinforcing buttress layer on both sides of the stapled union of visceral tissue to visceral tissue. The reinforcement is generally carried out by placement of a buttress assembly comprising an elongate strip of biocompatible material onto the anvil and cartridge of a prior art surgical stapler device. After the biocompatible material is securely but releasingly engaged onto the jaws of the surgical stapler, the jaws are closed for insertion through a trocar into the abdominal cavity after which the jaws are opened. The user then approximates the jaws around the target sections of the tissue to be stapled and separated. The jaws are again compressed and locked into place after which, a second handle is pulled to fire the staples followed by deployment of a knife to cut the host tissue and buttress material between the staple lines. The release mechanism is then activated to release the jaws from the staple line. Jaws are withdrawn from the suture site, closed and then the stapler is removed through the trocar.

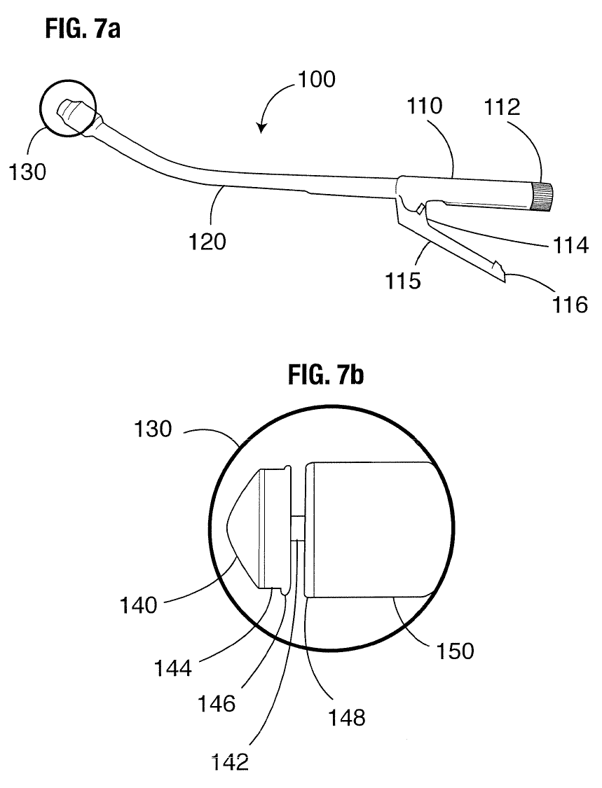

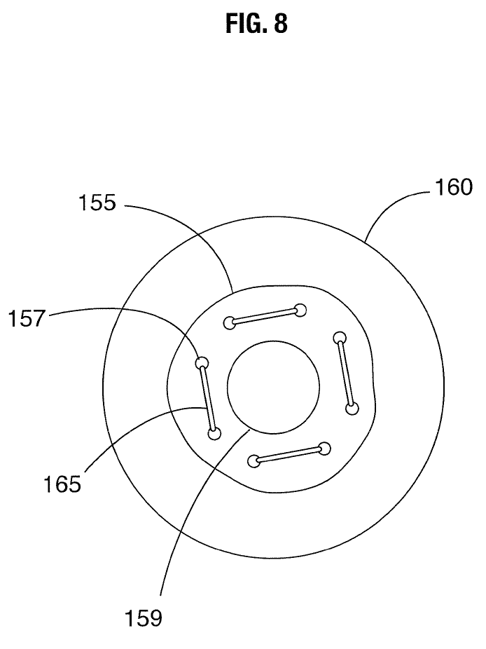

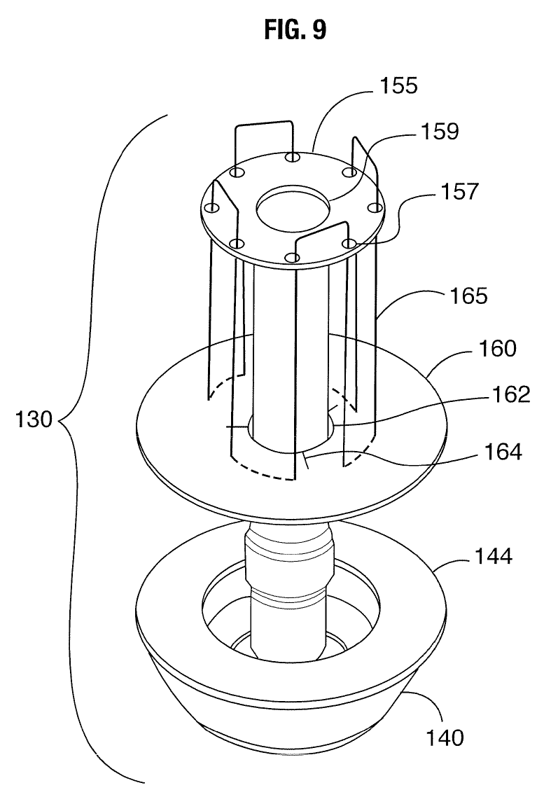

FIGS. 7a and 7b exemplify a prior art surgical circular stapler 100 having a proximal end provided with a handle 110 interconnected to a detachable anvil head assembly 130 at the distal end by a moisture-proof conduit 120. The anvil head assembly 130 comprises shaft 142 cooperating with a leading head portion 140 which facilitates insertion of the stapler 100 into a hollow organ or tissue section, and a cutting washer 146 interposed a first compression surface 144. The distal end of the conduit 120 is enlarged to form a cylindrical cartridge 150 configured for receiving and housing surgical staples. The cylindrical cartridge 150 is fitted with a fixed-in-place second compression surface 148 opposite the first compression surface provided within the anvil head assembly 130. The surgical staples are fired by squeezing an actuating lever 115 toward the handle 110 which results in the release of a trigger mechanism (not shown) housed within the conduit 120. The firing force is controllable by an adjusting knob 112 located at the proximal end of the stapler 100 cooperating with the trigger mechanism. A safety catch 116 is commonly provided to ensure the staples are not accidentally fired.

An exemplary embodiment of the present invention is shown in FIGS. 5 and 6 and provides a generally circular buttress disc 160 comprising a suitable biocompatible material, examples of which include synthetic base material such as ePTFE, VICRYL®, DEXON®, TEFLON®, and collagen-absorbable hemostat. Alternatively, stabilized naturally occurring materials such as a pericardium material, and other such materials may be used. The buttress disc 160 is provided with an aperture 162 positioned about the centre of the disc 160, said aperture 162 configured for slidingly cooperating with the shaft 142 of the anvil head assembly 130 of the stapler 100. The buttress disc 160 may optionally be provided with at least one, and preferably a plurality of slits 164 extending inward from aperture 162 to enable sliding cooperation of the buttress disc 160 with shafts having different diameters. The buttress disc 160 is attached to an orientating disc 155 provided with a plurality of bores 157 therethrough, by suturing the disc 160 to the bores 157 with a suitable running suture material 165. The orientating disc 155 is configured to position and maintain the buttress disc 160 in a coplanar orientation relative to the cartridge 150 and anvil head assembly 130. It is preferable that the outer diameter of the buttress disc 160 is slightly larger than the outer diameters of the first and second compression surfaces 144 and 148.

The orientating disc 155 to which a buttress disc 160 is sutured, is slid over the shaft 142 of the anvil head assembly 130 until the orientating disc is adjacent the first compression surface 144, after which the cutting washer 142 is slid over the shaft 142 until the cutting washer 142 is adjacent the orientating disc 155, thereby producing a loaded anvil head assembly 130. The loaded anvil head assembly 130 is then inserted head portion 140 first into the distal portion of a severed tubular tissue until a sufficient length of the severed tubular tissue extends beyond the shaft 142 to enable securing of the end of the tissue to the shaft 142 with a suture material. The severed end of the tubular tissue opposite the distal end is tied-off with a suture material after which, the distal end of the shaft 120 of the surgical stapler 100 is inserted into the proximal end of the severed tubular tissue until the second compression surface 148 fixed to the cylindrical cartridge 150 abuts the tied-off end of the tissue section. The shaft 142 of the anvil head assembly 130 is then maneuvered against the end of the cylindrical cartridge 150 thereby contacting the opposing tied-off ends of the severed tissues to be rejoined. The actuating lever 115 is then compressed against the handle 110 resulting in the firing of staples in a circular pattern after which they are compressed between the first and second compression surfaces 144 and 148 thereby conjoining the two severed tubular tissue sections. The cutting washer 146 is then activated to severe the opposing tied ends from the conjoined tissues thereby cutting out the centre of the conjoined tissue sections and a center portion of the buttress disc 160, thereby providing a conjoined continuous tubular tissue with the buttress material reinforcing the staple line against the inner surface of the conjoined tubular tissue. Those skilled in these arts will understand that it is optional, if so desired, to reinforce the staple line against the outer surface of the conjoined tissue by providing a second orientating disc 155 to which a buttress disc 160 is sutured, adjacent the second compression surface 148 (not illustrated) prior to inserting the distal end of the shaft 120 of the surgical stapler 100 into the proximal end of the severed tubular tissue against the tied-off end of the tissue section.

In view of numerous changes and variations that will be apparent to persons skilled in the art, the scope of the present invention is to be considered limited solely by the appended claims.

What is claimed is:

1. A surgical buttress-dispensing assembly configured for releasably engaging a surgical stapling device, the buttress-dispensing assembly comprising:
a one-piece cartridge for receiving and engaging therein an elongate buttress strip, said cartridge configured to slidingly communicate with and demountably engage the jaws of a surgical stapler; and
a carrier configured to slidingly receive, engage, and discharge therefrom the cartridge.

2. A surgical buttress-dispensing assembly according to claim 1, wherein the cartridge comprises a cylindrical end portion wherefrom extends a pair of opposed elongate semi-circular sleeves.

3. A surgical buttress-dispensing assembly according to claim 2, wherein the cartridge comprises a cylindrical end portion wherefrom extends a pair of opposed elongate semi-circular sleeves wherein one of said sleeves is configured to demountably engage a first jaw of a surgical stapler, and the other of said sleeves is configured to demountably engage a second jaw of said surgical stapler.

4. A surgical buttress-dispensing assembly according to claim 2, wherein said pair of opposed elongate semi-circular sleeves extends from a bore provided therethrough said cylindrical end portion.

5. A surgical buttress-dispensing assembly according to claim 2, wherein said pair of opposed elongate semi-circular sleeves are provided with elongate side edges adapted for engaging the elongate sides of an elongate buttress strip.

6. A surgical buttress-dispensing assembly according to claim 2, wherein said elongate side edges of said pair of opposed elongate semi-circular sleeves are provided with a plurality of forward-projecting fingers configured for engaging and retaining thereon edge portions of an elongate buttress strip.

7. A surgical buttress-dispensing assembly according to claim 2, wherein said carrier is configured to receive therein and demountably engage therewith one of said sleeves.

8. A surgical buttress-dispensing assembly according to claim 7, wherein said carrier comprises an elongate tube provided with a tang approximate a first end, said tang depending away from said first end.

9. A surgical buttress-dispensing assembly according to claim 7, wherein said carrier is configured to receive therein and demountably engage therewith an end of one of said sleeves opposite the cylindrical end portion of said cartridge.

10. A surgical buttress-dispensing assembly according to claim 3, further provided with an elongate buttress strip mounted onto said pair of opposed elongate semi-circular sleeves wherein a first end of the buttress strip is mounted onto one of said opposed elongate semi-circular sleeves and depends toward the cylindrical portion of said cartridge, and the other end of the buttress strip is mounted onto the other of said opposed elongate semi-circular sleeves and depends toward the cylindrical portion of said cartridge and is conjoined to said first end of said buttress strip.

11. A surgical buttress-dispensing assembly according to claim 10, wherein said elongate buttress strip comprises a biocompatible material.

12. A surgical buttress-dispensing assembly according to claim 11, wherein said elongate buttress strip is a synthetic base material selected from the group comprising polytetrafluoroethylene, expanded polytetrafluoroethylene, a periodontal mesh prepared from bioabsorbable copolymers derived from glycolic acid and lactic acid, polyglycolic acid, and collagen-absorbable hemostat.

13. A surgical buttress-dispensing assembly according to claim 11, wherein said elongate buttress strip is a stabilized naturally occurring material.

14. A surgical buttress-dispensing assembly according to claim 14, wherein said buttress strip is a stabilized pericardium.

15. A surgical buttress-dispensing cartridge configured for releasably engaging a surgical stapling device, the buttress dispensing cartridge comprising:
a cylindrical base portion;
a pair of opposing elongate fingers extending therefrom the cylindrical base portion, each finger comprising an arched bridge section wherefrom extend opposing upward and outward extending channels, each channel configured for receiving and engaging therein a portion of an elongate buttress strip;
wherein each finger is configured to slidingly communicate with a jaw of a surgical stapler.

16. A surgical buttress-dispensing cartridge according to claim 15, wherein said pair of opposing elongate fingers extend therefrom a bore provided therethrough said cylindrical base portion.

17. A surgical buttress-dispensing cartridge according to claim 15, wherein each of said channels is provided with a gripping means for engaging therewith a portion of a biocompatible buttress material.

18. A surgical buttress-dispensing cartridge according to claim 15, wherein said cartridge is further provided with an annular carrier configured for slidingly receiving therein and releasably engaging an end of one of said fingers.

19. A surgical buttress-dispensing cartridge according to claim 15, wherein said cartridge is further configured for carrying thereon and therewithin an elongate buttress strip, and for urging opposing side edges of said elongate buttress strip into a mounted engagement with said pair of opposing elongate fingers.

20. A surgical buttress-dispensing cartridge according to claim 15, further provided with an elongate buttress strip mounted onto said pair of opposed elongate fingers wherein a first end of the buttress strip is mounted onto one of said opposed elongate fingers and depends toward the cylindrical portion of said cartridge, and the other end of the buttress strip is mounted onto the other of said opposed elongate fingers and depends toward the cylindrical portion of said cartridge and is conjoined to said first end of said buttress strip.

21. A surgical buttress-dispensing cartridge according to claim 20, wherein said elongate buttress strip comprises a biocompatible material.

22. A surgical buttress-dispensing assembly according to claim 20, wherein said elongate buttress strip is a synthetic base material selected from the group comprising polytetrafluoroethylene, expanded polytetrafluoroethylene, a periodontal mesh prepared from bioabsorbable copolymers derived from glycolic acid and lactic acid, polyglycolic acid, and collagen-absorbable hemostat.

23. A surgical buttress-dispensing assembly according to claim 20, wherein said elongate buttress strip is a stabilized naturally occurring material.

24. A surgical buttress-dispensing assembly according to claim 23, wherein said buttress strip is a stabilized pericardium.

26. A surgical buttress-dispensing apparatus for cooperating with a surgical circular stapler, said buttress apparatus comprising:
a biocompatible buttress material having a middle portion provided with an aperture therethrough;
a circular buttress support disc provided with a concentric aperture therethrough, said support disc configured to sliding cooperate with an anvil head assembly of said surgical circular stapler; and
a device for threadably attaching said buttress material to said support disc.

27. A surgical buttress-dispensing assembly according to claim 26, wherein said elongate buttress strip is a synthetic base material selected from the group comprising polytetrafluoroethylene, expanded polytetrafluoroethylene, a periodontal mesh prepared from bioabsorbable copolymers derived from glycolic acid and lactic acid, polyglycolic acid, and collagen-absorbable hemostat.

28. A surgical buttress-dispensing assembly according to claim 26, wherein said elongate buttress strip is a stabilized naturally occurring material.

29. A surgical buttress-dispensing assembly according to claim 26, wherein said buttress strip is a stabilized pericardium.

30. A surgical buttress assembly configured for cooperating with a surgical circular stapler, said buttress assembly comprising:
a circular buttress support disc provided with a concentric aperture therethrough, said support disc configured to sliding cooperate with an anvil; and
a biocompatible buttress material threadably attached to said support disc.

31. A surgical buttress-dispensing assembly according to claim 30, wherein said elongate buttress strip is a synthetic base material selected from the group comprising polytetrafluoroethylene, expanded polytetrafluoroethylene, a periodontal mesh prepared from bioabsorbable copolymers derived from glycolic acid and lactic acid, polyglycolic acid, and collagen-absorbable hemostat.

32. A surgical buttress-dispensing assembly according to claim 30, wherein said elongate buttress strip is a stabilized naturally occurring material.

33. A surgical buttress-dispensing assembly according to claim 32, wherein said buttress strip is a stabilized pericardium.
